(19) United States
(12) Patent Application Publication
(10) Pub. No.: US 2024/0299172 A1
(43) Pub. Date: Sep. 12, 2024

(54) ANNULUS-REDUCING SYSTEM HAVING INDEPENDENT ANCHOR LAUNCHERS

(71) Applicant: EDWARDS LIFESCIENCES CORPORATION, Irvine, CA (US)

(72) Inventor: David Alon, Zichron Yaacov (IL)

(21) Appl. No.: 18/664,243

(22) Filed: May 14, 2024

Related U.S. Application Data
(63) Continuation of application No. 17/323,644, filed on May 18, 2021, now Pat. No. 12,023,247.
(60) Provisional application No. 63/027,640, filed on May 20, 2020.

(51) Int. Cl. A61F 2/24; A61M 25/01

(52) U.S. Cl. CPC A61F 2/2466; A61M 25/0105; A61F 2220/0016; A61M 2025/0166

(57) ABSTRACT

Using a catheter, a device is delivered to the heart of a subject. The device includes a cord and multiple anchors distributed along the cord. Subsequently, the device is arranged in an arrangement at the tissue such that the cord is positioned and laid out correspondingly to the tissue. While the device remains in the arrangement, (i) a first subset of the anchors which are positioned for successful anchoring is identified, and (ii) subsequent to the identifying, for each of the anchors of the first subset, a respective anchor launcher is actuated to drive the anchor into the tissue. Subsequently, for each of the anchors that remain not driven into the tissue, (i) the anchor is positioned for successful anchoring, and (ii) a respective anchor launcher is actuated to drive the anchor into the tissue. Other embodiments are also disclosed.

### CROSS REFERENCE TO RELATED APPLICATIONS

The present application is a Continuation of U.S. patent application Ser. No. 17/323,644 to Alon et al., filed May 18, 2021; which claims the benefit of Provisional U.S. Patent Application No. 63/027,640, filed May 20, 2020, which is incorporated herein by reference in its entirety.

### BACKGROUND

U.S. Pat. Nos. 9,517,130, 10,143,553, 10,206,776, and 10,575,952, each of which is incorporated by reference, describes a variety of approaches for (a) affixing a cord to a cardiac valve annulus or to adjacent tissue (e.g., leaflets of the valve) using a plurality of anchors and (b) getting the cord in position before the anchors are driven into the tissue. After the cord has been affixed, tissue healing strengthens the bond between the annulus and the anchors over time (e.g., 4-8 weeks). In some cases, tissue healing also strengthens a bond between the annulus and a sleeve that promotes tissue ingrowth that surrounds the cord. After the bond has been strengthened, the cord can be used to reduce the size of the annulus.

As best described in U.S. Pat. No. 10,206,776, each of the anchors that is driven into the tissue has a corresponding anchor launcher, and each of the anchor launchers has its own individual actuator (e.g., a pull wire). The '776 patent also describes a mechanism for triggering all of the anchor launchers by pulling on a set of pull wires that serve as the actuators for the anchor launchers. This can be done either sequentially (i.e., by triggering one anchor launcher at a time in rapid succession) or simultaneously (i.e., by triggering all of the anchor launchers simultaneously). But in either situation, the mechanism for triggering the anchor launchers always triggered each and every one of the anchor launchers.

In view of this arrangement, the cord, the anchors, and the anchor launchers in the '776 patent had to be positioned very precisely before the anchors were launched. For if one or more anchors was not positioned close enough to the target tissue at the moment those anchors were launched out of the respective anchor launchers, those anchors would not become properly embedded in the annulus or the adjacent tissue.

### SUMMARY OF THE INVENTION

One aspect of the invention is directed to a first method of affixing a device to a cardiac valve annulus. The first method comprises delivering a flexible cord, N anchors, and N anchor launchers to the vicinity of the annulus via a catheter, wherein N is an integer greater than 4, wherein the N anchors are distributed about the flexible cord, wherein each of the N anchors is affixed with respect to the flexible cord, and wherein each of the N anchor launchers is configured to, upon actuation, drive a respective one of the N anchors into the annulus or tissue adjacent to the annulus. Subsequent to the delivering, a position and layout of the flexible cord is adjusted via the catheter until the position and layout of the flexible cord corresponds the annulus. Subsequent to the adjusting, a first subset of the N anchors which are positioned such that the respective anchor launcher can successfully drive the respective anchor into the annulus or tissue adjacent to the annulus are identified, wherein the first subset includes fewer than N anchors. Subsequent to the identifying, a first subset of the N anchor launchers that corresponds to the first subset of the N anchors is actuated, so that the first subset of the N anchor launchers drive the first subset of the N anchors into the annulus or tissue adjacent to the annulus. The first method also comprises introducing a manipulating tool via the catheter to the vicinity of the annulus. Subsequent to the actuating of the first subset of the N anchor launchers, for each of the N anchors that have not yet been driven into the annulus or tissue adjacent to the annulus, (a) the manipulating tool is used to move the anchor to a position where the respective anchor launcher can successfully drive the anchor into the annulus or tissue adjacent to the annulus, and (b) the respective anchor launcher is actuated so that the respective anchor launcher drives the anchor into the annulus or tissue adjacent to the annulus.

In some instances of the first method, each of the N anchors is slidably affixed with respect to the flexible cord and is configured so that when a respective anchor is driven into the annulus or tissue adjacent to the annulus by a respective anchor launcher, the respective anchor will slide from an initial position to a final position at which the respective anchor fastens a respective portion of the flexible cord to the annulus or to the tissue adjacent to the annulus.

In some instances of the first method, the identifying step comprises at least one of echo imaging and flouro imaging. In some instances of the first method, the identifying step comprises determining whether a contact probe that is affixed to each of the anchor launchers is making contact with tissue. In some instances of the first method, the actuating of the first subset of the N anchor launchers comprises actuating the entire first subset of the N anchor launchers substantially simultaneously. In some instances of the first method, the step of adjusting a position and layout of the flexible cord comprises inflating a balloon. In some instances of the first method, the first subset includes at least two anchors.

Another aspect of the invention is directed to a second method of affixing a device to a cardiac valve annulus. The second method comprises delivering a flexible cord, N anchors, and N anchor launchers to the vicinity of the annulus via a catheter, wherein N is an integer greater than 4, wherein the N anchors are distributed about the flexible cord, wherein each of the N anchors is affixed with respect to the flexible cord, and wherein each of the N anchor launchers is configured to, upon actuation, drive a respective one of the N anchors into the annulus or tissue adjacent to the annulus. Subsequent to the delivering, a position and layout of the flexible cord is adjusted via the catheter until the position and layout of the flexible cord corresponds the annulus. Subsequent to the adjusting, a first subset of the N anchors which are positioned such that the respective anchor launcher can successfully drive the respective anchor into the annulus or tissue adjacent to the annulus is identified, wherein the first subset includes fewer than N anchors. Subsequent to the identifying, a first subset of the N anchor launchers that corresponds to the first subset of the N anchors is actuated, so that the first subset of the N anchor launchers drive the first subset of the N anchors into the annulus or tissue adjacent to the annulus. Subsequent to the actuating of the first subset of the N anchor launchers, for each of the N anchors that have not yet been driven into the annulus or tissue adjacent to the annulus, (a) catheter steering techniques are used to adjust a position of a respective corresponding anchor launcher to a position where the respective corresponding anchor launcher can successfully drive the anchor into the annulus or tissue adjacent to the annulus, and (b) the respective corresponding anchor launcher is actuated so that the respective corresponding anchor launcher drives the anchor into the annulus or tissue adjacent to the annulus.

In some instances of the second method, steps (a) and (b) are performed at least twice, and steps (a) and (b) are repeated until all of the anchors have been driven into the annulus or tissue adjacent to the annulus.

In some instances of the second method, each of the N anchors is slidably affixed with respect to the flexible cord and is configured so that when a respective anchor is driven into the annulus or tissue adjacent to the annulus by a respective anchor launcher, the respective anchor will slide from an initial position to a final position at which the respective anchor fastens a respective portion of the flexible cord to the annulus or to the tissue adjacent to the annulus.

In some instances of the second method, the identifying step comprises at least one of echo imaging and flouro imaging. In some instances of the second method, the identifying step comprises determining whether a contact probe that is affixed to each of the anchor launchers is making contact with tissue. In some instances of the second method, the actuating of the first subset of the N anchor launchers comprises actuating the entire first subset of the N anchor launchers substantially simultaneously. In some instances of the second method, the first subset includes at least two anchors.

Another aspect of the invention is directed to a third method of affixing a device to a cardiac valve annulus. The third method comprises delivering a flexible cord, N anchors, and N anchor launchers to the vicinity of the annulus via a catheter, wherein N is an integer greater than 4, wherein the N anchors are distributed about the flexible cord, wherein each of the N anchors is affixed with respect to the flexible cord, and wherein each of the N anchor launchers is configured to, upon actuation, drive a respective one of the N anchors into the annulus or tissue adjacent to the annulus. The third method also comprises introducing a manipulating tool via the catheter to the vicinity of the annulus. For each of the N anchors in turn, (a) the manipulating tool is used to move the anchor to a position where the respective anchor launcher can successfully drive the anchor into the annulus or tissue adjacent to the annulus, and (b) the respective anchor launcher is actuated so that the respective anchor launcher drives the anchor into the annulus or tissue adjacent to the annulus.

In some instances of the third method, each of the N anchors is slidably affixed with respect to the flexible cord and is configured so that when a respective anchor is driven into the annulus or tissue adjacent to the annulus by a respective anchor launcher, the respective anchor will slide from an initial position to a final position at which the respective anchor fastens a respective portion of the flexible cord to the annulus or to the tissue adjacent to the annulus.

Some instances of the third method further comprise using at least one of echo imaging and flouro imaging to determine the position of the anchors while the manipulating tool is being used to move the anchors. Some instances of the third method further comprise determining whether a contact probe that is affixed to each of the anchor launchers is making contact with tissue. Some instances of the third method further comprise, subsequent to the delivering, adjusting a position and layout of the flexible cord via the catheter until the position and layout of the flexible cord corresponds the annulus.

In some instances of the third method, the step of adjusting a position and layout of the flexible cord comprises inflating a balloon.

### BRIEF DESCRIPTION OF THE DRAWINGS

FIG. 1 depicts one example of an approach for implementing individually-controllable actuators.

FIGS. 2A and 2B depict one example of a mechanism for implementing any given one of the actuators depicted in FIG. 1.

FIG. 3 depicts an example of an annulus-constricting device positioned next to a cardiac valve annulus prior to launching of the anchors, where only 8 of the 10 anchor launchers are positioned for launching their respective anchors.

FIGS. 4A, 4B depict two detail views of the FIG. 3 embodiment after eight anchors have been launched and two anchors have not been launched.

FIG. 5 depicts an overall view of the FIG. 3 embodiment after eight anchors have been launched and two anchors have not been launched.

FIG. 6 depicts a manipulating tool that is being used to reposition the ninth anchor.

FIG. 7 depicts the manipulating tool that is being used to reposition the tenth anchor.

FIG. 8 depicts the FIG. 3 embodiment after the manipulating tool has been withdrawn.

FIG. 9 depicts the FIG. 3 embodiment after the anchor launchers have been withdrawn.

FIG. 10 depicts a detailed view of the manipulating tool depicted in FIGS. 6 and 7.

FIGS. 11A and 11B depict an alternative manipulating tool that may be used instead of the tool shown in FIG. 10.

FIGS. 12A and 12B depict another alternative manipulating tool that may be used instead of the tool shown in FIG. 10.

FIG. 13 depicts another alternative manipulating tool that may be used instead of the tool shown in FIG. 10.

FIG. 14 depicts another example of an approach for implementing individually-controllable actuators.

FIG. 15 depicts an example of another annulus-constricting device positioned next to a cardiac valve annulus prior to launching of the anchors, where only seven of the anchor launchers are positioned for launching their respective anchors.

FIG. 16 depicts an overall view of the FIG. 15 embodiment after seven anchors have been launched and three anchors have not been launched.

Various embodiments are described in detail below with reference to the accompanying drawings, wherein like reference numerals represent like elements.

### DESCRIPTION OF THE PREFERRED EMBODIMENTS

The embodiments described herein significantly relax the requirements to position the cord, the anchors and the anchor launchers precisely before the anchors are launched. This is accomplished by positioning the cord up against the annulus when the anchors are ready to launch, then identifying which anchors are properly positioned so that they can be successfully driven into the annulus or adjacent tissue. This identification may be implemented, for example, using echo imaging, fluoro imaging, or contact probes that are affixed to each of the anchor launchers.

Next, only those anchor launchers that are in a position where the corresponding anchors can be successfully driven into the annulus or adjacent tissue are actuated. This will drive the majority of the anchors into the annulus or adjacent tissue. But a small number of anchors will not be launched, and will remain in their respective anchor launchers. In some embodiments, a manipulator tool is then introduced, and is used to move each of the unlaunched anchors to a position where it can be successfully driven into the annulus. In other embodiments, each of the unlaunched anchors is moved to a position where it can be successfully driven into the annulus using catheter-steering techniques.

Unlike the prior art approach described in U.S. Pat. No. 10,206,776, in which a single user-operated control triggers all of the actuators, the actuators in the embodiments described herein can be controlled individually. Assume, for example, that the anchor launchers described in connection with FIGS. 54A and 54B of U.S. Pat. No. 10,206,776, which is incorporated herein by reference, are being used. Those anchor launchers are triggered by pulling on an actuation wire, and an individual actuation wire is provided for each anchor launcher.

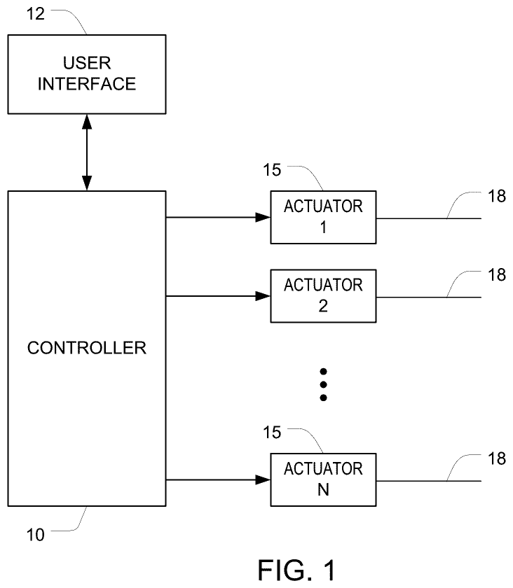

FIG. 1 depicts one example of an approach for implementing individually-controllable actuators. In this approach, an individually controllable actuator 15 is provided for each anchor launcher, and each of those individually controllable actuators 15 pulls on a respective one of the actuation wires 18. The number of actuators 15 will correspond to the number of anchors that are used to affix the cord to the cardiac valve annulus or to adjacent tissue. For example, in those embodiments where N anchors are used to affix the cord (where N is an integer), there will be a corresponding number (N) of anchor launchers, and each of those N anchor launchers will have its own individual actuation wire 18.

Because the actuators 15 are individually controllable, it now becomes possible to actuate any desired subset of the N anchor launchers without actuating the rest of the anchor launchers. Assume, for example, that N=8 and the operator has decided to launch anchors #1, #2, #3, #5, #6, and #8, but not to launch anchors #4 and #7. This decision may be based, for example, on echo imaging, fluoro imaging, or another modality of imaging. The user informs the system which anchors should be launch via user interface 12, and the user interface 12 conveys the list of anchors to be launched to the controller 10. Alternatively, the decision of which launchers should be launched may be based on information received from a set of N contact sensors (not shown), each of which is configured to detect when the distal end of a respective anchor launcher is making contact with tissue. When contact sensors are provided, the contact sensors convey the list of anchors that should be launched to the controller 10.

The user interface 12 has a “launch” button (or other user control). When the user presses the “launch” button, the user interface 12 sends a signal to the controller 10, requesting launching of the previously identified subset of anchors (in the current example, this would be anchors #1, #2, #3, #5, #6, and #8). The controller 10 effectuates launching of the identified subset of anchors by sending an appropriate command to actuators (15) #1, #2, #3, #5, #6, and #8, but not sending that command to actuators (15) #4 and #7. Upon receiving the command, only actuators (15) #1, #2, #3, #5, #6, and #8 will pull their respective actuation wires 18. Those actuation wires 18 will actuate only anchor launchers #1, #2, #3, #5, #6, and #8, and those anchor launchers will launch their respective anchors into the cardiac valve annulus or adjacent tissue. Meanwhile, because anchor launchers #4 and #7 have not been actuated, anchors #4 and #7 will remain unlaunched (for the time being).

At a subsequent time, the unlaunched anchors can be driven into the cardiac valve annulus or adjacent tissue by actuating their respective anchor launchers individually. The controller 10 can accomplish this, for example, as follows: First, the controller 10 sends an appropriate command to actuator (15) #4 only. Upon receiving the command, only actuator #4 will pull its respective actuation wire 18. That actuation wire will actuate only anchor launcher #4, which will launch anchor #4 into the cardiac valve annulus or adjacent tissue. Subsequently, the controller 10 sends an appropriate command to actuator #7 only. Upon receiving the command, only actuator (15) #7 will pull its respective actuation wire 18. That actuation wire will actuate only anchor launcher #7, which will launch anchor #7 into the cardiac valve annulus or adjacent tissue.

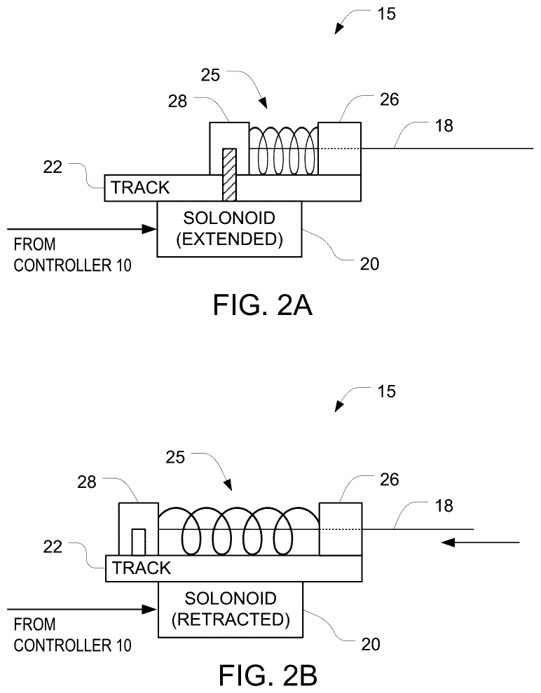

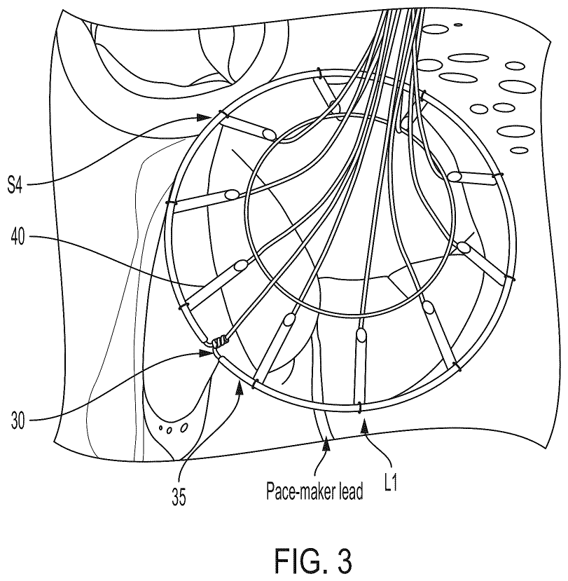

FIGS. 2A and 2B depict one example of a mechanism for implementing any given one of the actuators 15 depicted in FIG. 1. In this example, the actuator 15 relies on a spring 25 to pull the actuation wire 18. Initially, as seen in FIG. 2A, the spring 25 is held in a compressed state between base 26 and cap 28. The base 26 is permanently affixed to a track 22, and the cap 28 is slidably connected to the track 22. Solenoid 20 has a plunger that, in its extended state, prevents the cap 28 from moving. The actuation wire 18 is affixed to the cap 28 so that movement of the cap 28 to the left will pull the actuation wire 18 to the left. As long as the cap 28 remains in the position depicted in FIG. 2A, the actuation wire 18 will not be pulled; the anchor launchers will not be actuated; and the anchors will remain in position within their respective anchor launchers.

Turning now to FIG. 2B, when the controller 10 (depicted in FIG. 1) sends an appropriate signal to the solenoid 20, the plunger of the solenoid retracts. Once the plunger retracts, nothing remains to prevent the cap 28 from moving with respect to the track 22, and the spring 25 is now free to expand to its relaxed state. The spring 25 pushes the cap 28 to the left; and because the actuation wire 18 is connected to the cap 28, the cap 28 will pull the actuation wire 18 to the left. This will actuate the corresponding anchor launcher so that it ejects the corresponding anchor and drives it into the tissue.

Of course, the solenoid-based embodiment depicted in FIGS. 2A and 2B is only one example of a suitable mechanism for implementing the actuators 15 depicted in FIG. 1; and a wide variety of alternative approaches for individually actuating each of the anchor launchers 40 will be apparent to persons skilled in the relevant art.

FIGS. 3-9 depict an example of affixing an annulus-constricting device to a cardiac valve annulus. In this example, N=10, so there are 10 anchors 50 distributed about the flexible cord 30, and each of those 10 anchors 50 is slidably affixed with respect to the flexible cord 30. In the embodiment depicted in FIGS. 3-9, an optional sleeve 35 made from a material that accepts tissue ingrowth surrounds the flexible cord 30.

Examples of how to construct the anchors 50 and how to slidably affix the anchors 50 to the flexible cord 30 can be found, for example, in U.S. Pat. Nos. 9,517,130 and 10,206,776, each of which is incorporated herein by reference in its entirety. The slidable affixation of the anchors to the cord may be direct (e.g., when the cord is threaded directly through a slot in each of the anchors) or indirect (e.g., when the cord is threaded through an eyelet, and a slot in each of the anchors slides with respect to the eyelet). There are also 10 anchor launchers 40, and each of those 10 anchor launchers 40 is configured to, upon actuation, drive a respective one of the 10 anchors 50 into the annulus or tissue adjacent to the annulus. Examples of suitable anchor launchers 40 can be found, for example, in U.S. Pat. No. 10,206,776.

The flexible cord 30, the anchors 50, and the anchor launchers 40 are delivered to the vicinity of the annulus via a catheter (e.g., as described in U.S. Pat. No. 10,575,952, which is incorporated herein by reference in its entirety). Subsequent to the delivering, the position and layout of the flexible cord 30 is adjusted via the catheter until the position and layout of the flexible cord 30 corresponds the annulus, as depicted in FIG. 3. Optionally, an inflatable balloon (not shown) positioned between the arms that support the anchor launchers 40 may be used to help put the flexible cord 30 into a shape that approximates the shape of the annulus. The adjusting of the position of the flexible cord 30 may be implemented by an operator (e.g., using conventional catheter steering techniques) based on visual feedback obtained, for example, using echo imaging or fluoro imaging. Alternatively or additionally, the adjusting of the position of the flexible cord 30 may be implemented by an operator based on signals received from a set of contact probes (not shown) which are positioned on each anchor launcher 40 and provide a real time indication of contact with the tissue (e.g., by sending an electrical signal to the controller 10 shown in FIG. 1).

In the example depicted in FIG. 3, only two anchor launchers 40 are NOT positioned such that the respective anchor launcher can successfully drive the respective anchor into the annulus or tissue adjacent to the annulus, because those two anchor launchers 40 are out of contact with corresponding tissue. This may occur, for example, due to surface variations like a small landing zone in the septal region (for the anchor launcher 40 labeled S4) or due to a pacemaker lead in a posterior region (for the anchor launcher 40 labeled L1). The remaining eight anchor launchers 40 ARE positioned such that the respective anchor launcher can successfully drive the respective anchor into the annulus or tissue adjacent to the annulus, because those eight anchor launchers 40 are in contact (or at least close enough) with corresponding tissue.

Subsequent to the position adjusting, a first subset of the 10 anchors 50 (i.e., those anchors which ARE positioned such that the respective anchor launcher can successfully drive the respective anchor into the annulus or tissue adjacent to the annulus) is identified. The first subset includes fewer than 10 anchors 50 (i.e., 8 anchors 50 in the illustrated embodiment). The identification may be accomplished, for example, using echo imaging, fluoro imaging, or based on signals received from the contact probes (not shown). In some preferred embodiments (including this example in which the first subset includes 8 anchors), the first subset includes at least two anchors. But in other embodiments, the first subset could include only a single anchor.

Subsequent to the identifying, a first subset of the anchor launchers (i.e., 8 anchor launchers 40 in the illustrated embodiment) that corresponds to the first subset of the anchors 50 is actuated. This causes the first subset of the anchor launchers 40 to drive the first subset of the anchors 50 into the annulus or tissue adjacent to the annulus. In the illustrated embodiment, the total number of anchor launchers 40 is 10, and the first subset includes 8 anchors 50. This means that only 2 anchors 50 have not been launched at this point in the procedure.

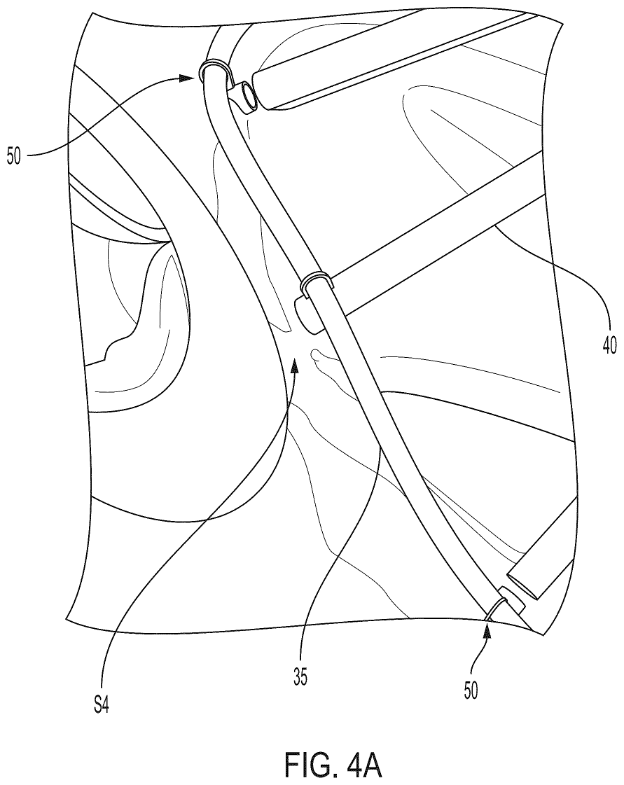

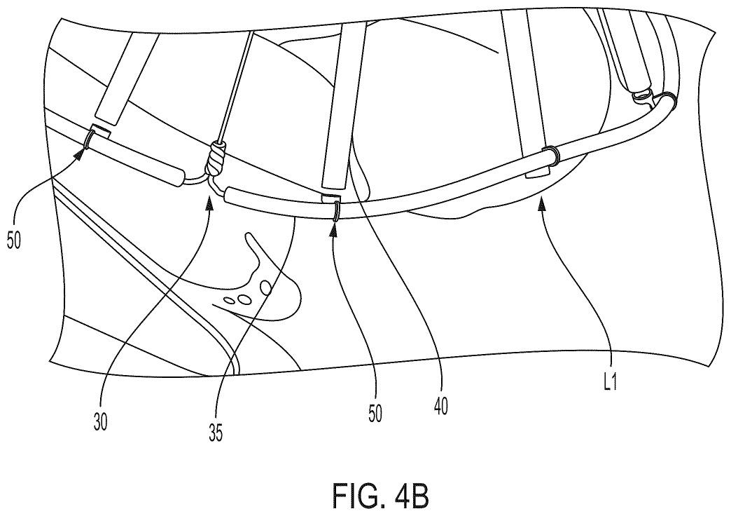

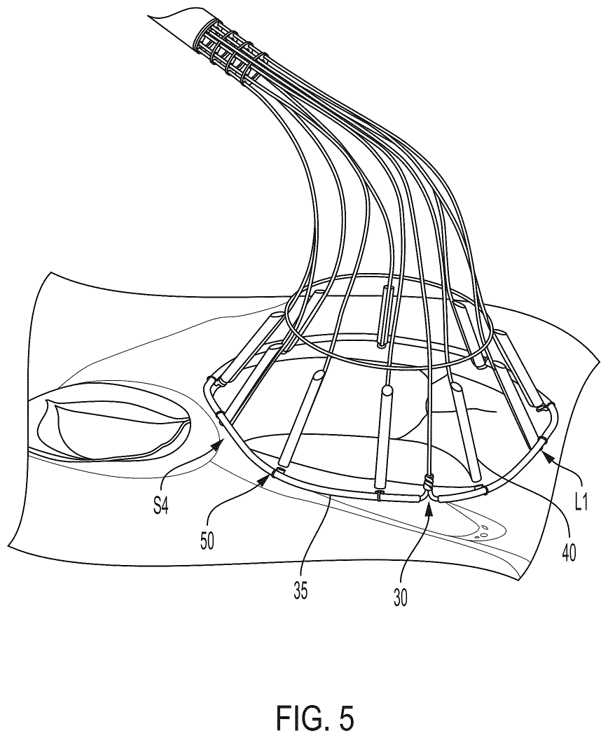

Turning now to FIGS. 4A, 4B, and 5 in the illustrated embodiment, the two unlaunched anchors 50 at this point in the procedure correspond to the anchor launchers 40 labeled L1 and S4. The remaining eight anchors 50 have been driven into the annulus or adjacent tissue by the corresponding anchor launchers 40. In those embodiments where a balloon (not shown) was used to help bring the flexible cord 30 into position, the balloon should be deflated and withdrawn at this point in the procedure.

At this point in the procedure, the scaffold of arms that support the anchor launchers may collapse (fully or partially) because most of the anchors 50 that are attached to the flexible cord 30 are no longer held in place by their respective anchor launchers 40. Alternatively, in some embodiments, the scaffold of arms may be made from a soft metal that retains its shape after the balloon is removed and the majority of the anchors 50 have been launched.

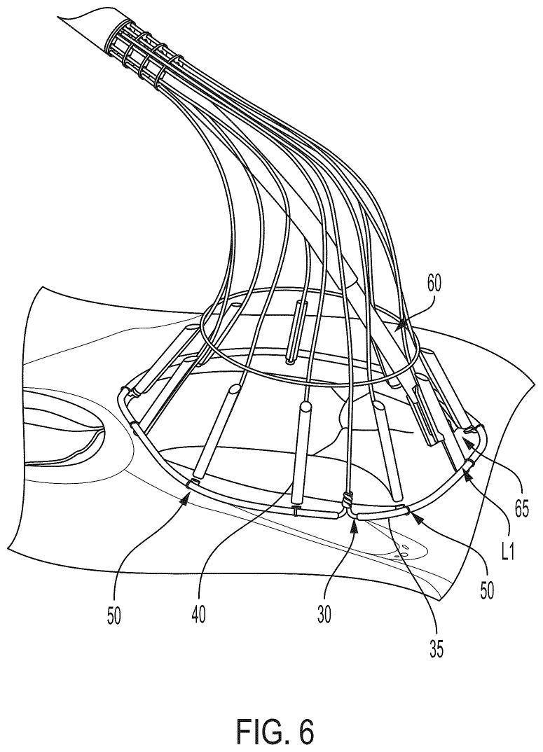

Next, as seen in FIG. 6, a manipulating tool 60 is introduced to the vicinity of the annulus via the catheter. The distal end of the manipulating tool 60 has a grabber 65 with jaws that can be opened and closed by the health practitioner. (As used herein, “distal” is further away from the practitioner who is operating the apparatus, and “proximal” is closer to the practitioner.) The grabber 65 is shaped and dimensioned to grab onto any of the anchor launchers 40 and subsequently manipulate the anchor launcher 40 to a desired position (i.e., to a position where the corresponding anchor 50 can be driven into the annulus or adjacent tissue). Manipulation of the position of the grabber 65 and the opening and closing of the jaws of the grabber 65 is controlled by the practitioner using a set of proximal controls (not shown).

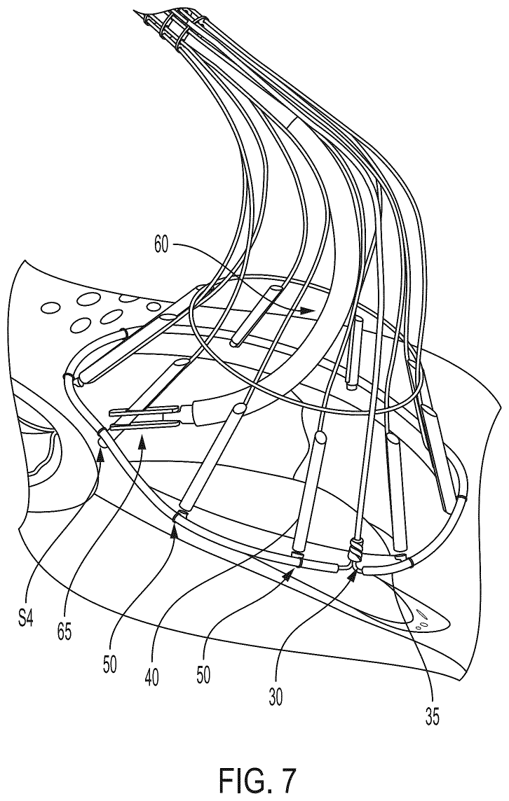

Subsequent to the actuating of the 8 anchor launchers 40 in the illustrated embodiment, for each of the 10 anchors 50 that have not yet been driven into the annulus or tissue adjacent to the annulus (i.e., for the 2 unlaunched anchors 50 in the illustrated embodiment), (a) the manipulating tool 60 is used to move the anchor to a position where the respective anchor launcher 40 can successfully drive the anchor 50 into the annulus or tissue adjacent to the annulus, and (b) the respective anchor launcher 40 is actuated so that the respective anchor launcher 40 drives the anchor 50 into the annulus or tissue adjacent to the annulus. For example, if only the anchors 50 corresponding to the anchor launchers 40 labeled L1 and S4 have not been launched. Steps (a) and (b) in this paragraph are first performed for the L1 anchor, as seen in FIG. 6. Then, steps (a) and (b) are performed for the S4 anchor, as seen in FIG. 7.

The movement of each anchor in step (a) may be implemented by an operator using the manipulating tool 60 based on visual feedback obtained, for example, using echo imaging or fluoro imaging. Alternatively or additionally, the operator may rely on feedback based on signals received from a contact probe (not shown) positioned on whichever anchor launcher 40 is being moved at any given instant. The contact probe provides a real time indication of contact with the tissue (e.g., by sending an electrical signal to the controller 10 shown in FIG. 1).

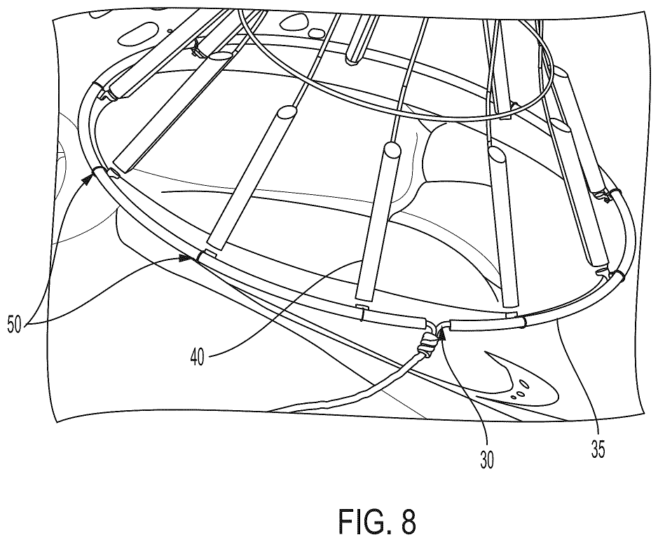

After steps (a) and (b) are performed for all of the anchors that were not launched in the initial batch (i.e., for L1 and S4), the manipulating tool 60 is withdrawn, as seen in FIG. 8. Finally, the anchor launchers 40 are withdrawn, leaving only the flexible cord 30, the optional sleeve 35 surrounding the flexible loop, and the anchors 50 (which have been driven into the annulus or adjacent tissue) behind in the patient's body, as seen in FIG. 9.

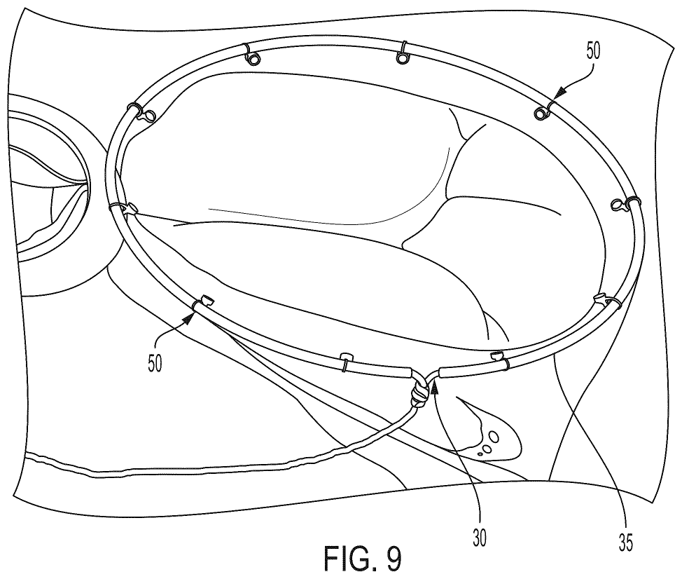

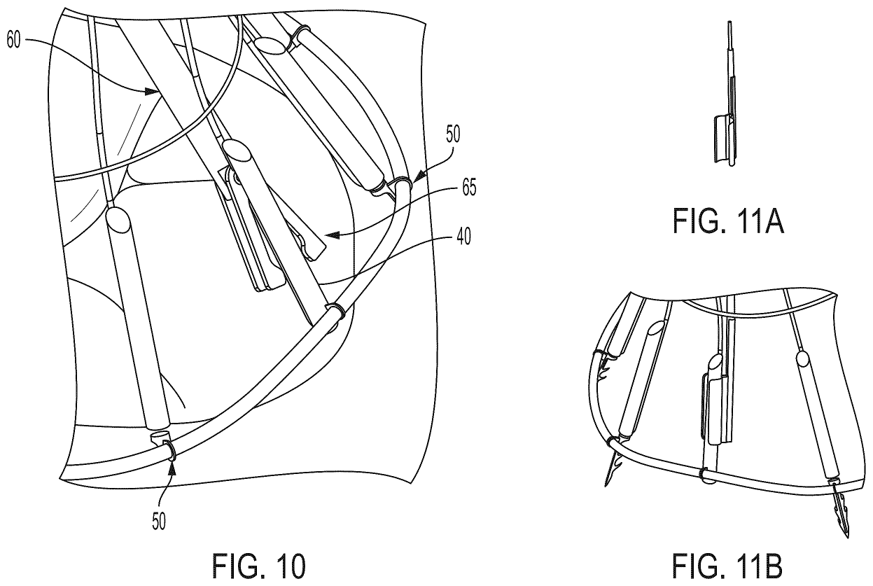

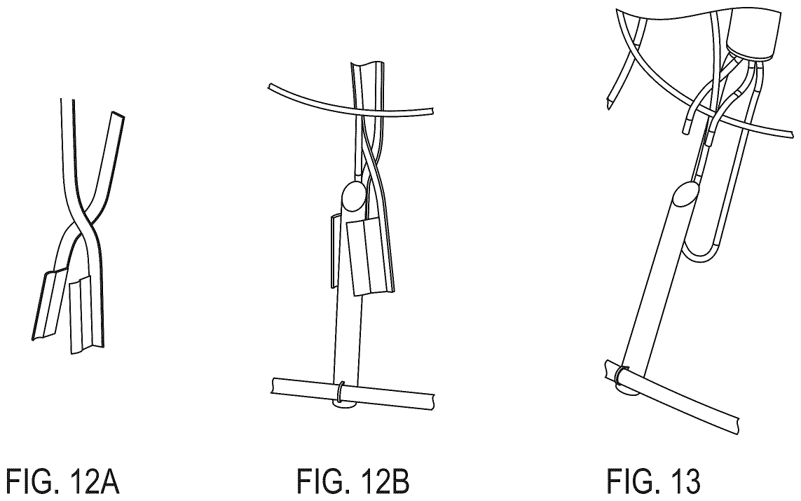

A wide variety of alternatives for implementing the grabber tool 65 can be readily envisioned, including but not limited to the configurations depicted in FIG. 10 (which has jaws that resemble pliers), FIGS. 11A and 11B (which has jaws that resemble a Venus fly trap), FIGS. 12A and 12B (which has jaws that resemble tongs), and FIG. 13 (which has an anchor-launcher holder that resembles a paper clip).

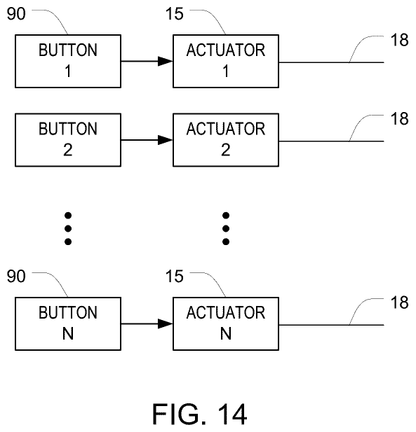

FIG. 14 is an example of another approach for individually actuating each of the anchor launchers 40. As in the FIG. 1-2 embodiment, an individually controllable actuator 15 is provided for each anchor launcher, and each of those individually controllable actuators 15 pulls on a respective one of the actuation wires 18. And as in the FIG. 1-2 embodiment, the number of actuators 15 will correspond to the number of anchors that are used to affix the cord to the cardiac valve annulus or to adjacent tissue.

In this FIG. 14 embodiment, each of the actuators 15 has its own individual “launch” button 90 (or other user control). And each of these launch buttons 90 is configured, e.g., via a suitable mechanical linkage, so that when the user presses any given launch button 90, the corresponding actuator 15 will pull its respective actuation wire 18. One suitable approach for implementing the actuators 15 is similar to the approach depicted above in connection with FIG. 2A-2B. But instead of using a solenoid with a plunger that, in its extended state, holds the cap 28 in place on the track 22, this FIG. 14 embodiment uses a pin (not shown) that in its initial extended state, holds the cap in place on the track. Pressing each button 90 causes (by operation of the mechanical linkage) the corresponding pin to retract, which has the same effect as retraction of the plunger in the FIG. 2A-2B embodiment.

In this embodiment, it is possible to actuate any desired subset of the N anchor launchers at any given time by pressing the corresponding launch buttons 90. Assume, for example, that there are 8 anchors and the user wants to launch anchors nos. 1-6 but not to launch anchors nos. 7-8. In this case, the user would press only buttons 90 nos. 1-6, which will cause the corresponding actuators 15 nos. 1-6 to pull their respective actuation wires. Those actuation wires 18 will actuate only anchor launchers nos. 1-6, but will not actuate anchor launcher nos. 7-8. At a subsequent time, the unlaunched anchors can be driven into the cardiac valve annulus or adjacent tissue by pressing the corresponding buttons 90 to actuate each of the remaining anchor launchers individually. Because this is similar to the situation described above in connection with the FIGS. 1-2 embodiment (in that independent control over launching is provided for each anchor), the examples described above in connection with FIGS. 3-13 apply with equal force to this FIG. 14 embodiment.

Note that in the embodiments described above in connection with FIGS. 1-14, (i) the position and layout of the flexible cord is adjusted via the catheter until the position and layout of the flexible cord corresponds the annulus, and (ii) the first subset of the N anchor launchers are actuated before the manipulating tool is introduced to the vicinity of the catheter. Thus, at least one of the anchors will be launched without relying on the manipulating tool.

In alternative embodiments, the manipulating tool is relied upon for the launching of ALL of the anchors. In these embodiments, the steps of identifying and actuating that occur prior to the introduction of the manipulating tool in the embodiments described above are omitted. And the step of adjusting a position and layout of the flexible cord via the catheter until the position and layout of the flexible cord corresponds the annulus can optionally also be omitted.

In these embodiments, the flexible cord, the N anchors, and the N anchor launchers are delivered to the vicinity of the annulus via the catheter as in the embodiments described above. The manipulating tool is introduced via the catheter to the vicinity of the annulus. Subsequently, for each of the N anchors in turn, (a) the manipulating tool is used to move the anchor to a position where the respective anchor launcher can successfully drive the anchor into the annulus or tissue adjacent to the annulus, and (b) the respective anchor launcher is actuated so that the respective anchor launcher drives the anchor into the annulus or tissue adjacent to the annulus. As a result of performing steps (a) and (b) for each of the N anchors in turn, all N anchors will be driven into the annulus or tissue adjacent to the annulus, thereby affixing the flexible cord to the annulus.

Optionally, echo imaging or flouro imaging may be used to determine the position of the anchors while the manipulating tool is being used to move the anchors. Optionally, a set of N contact probes, each of which is affixed to a respective anchor launcher, may be used to determine if each of the respective anchor launchers is making contact with tissue prior to actuating that anchor launcher.

Optionally, subsequent to delivering the flexible cord, the N anchors, and the N anchor launchers, the position and layout of the flexible cord may be adjusted via the catheter until the position and layout of the flexible cord corresponds the annulus. This step may optionally be implemented by inflating a balloon.

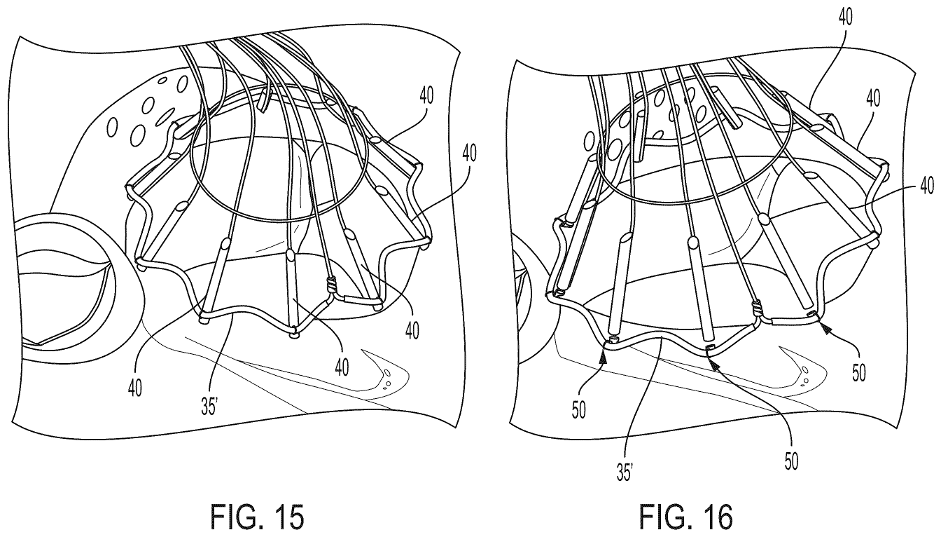

In the embodiments described above, the manipulating tool is used to move at least some of the anchor launchers to positions where they can successfully drive the anchor into the annulus or tissue adjacent to the annulus. In contrast, the embodiments described below in connection with FIGS. 15 and 16 move the anchor launchers to their desired position using only catheter steering techniques.

FIGS. 15 and 16 depict an example of affixing an annulus-constricting device to a cardiac valve annulus. In this example, N=10, so there are 10 anchors 50 distributed about the flexible cord 30, and each of those 10 anchors 50 is slidably affixed with respect to the flexible cord 30. In the embodiment depicted in FIGS. 15-16, an optional sleeve 35′ made from a material that accepts tissue ingrowth surrounds the flexible cord 30. The flexible cord 30, the optional sleeve 35, the optional balloon (not shown), the anchor launchers 40, and the anchors 50 in these embodiments are the same as the corresponding components in the FIGS. 3-5 embodiments described above, except that the segments of cord 30 and sleeve 35′ between any two given anchors 50 have additional slack.

All of these components 30, 35′, 40, 50 are delivered to the vicinity of the annulus via a catheter as described above in connection with FIGS. 3-5. Subsequent to the delivering, the position and layout of the flexible cord 30 is adjusted via the catheter (e.g., using catheter steering techniques) until the position and layout of the flexible cord 30 corresponds the annulus, as described above in connection with FIGS. 3-5 and as depicted in FIG. 15.

In the example depicted in FIG. 15, assume that only three anchor launchers 40 are NOT positioned such that the respective anchor launcher can successfully drive the respective anchor into the annulus or tissue adjacent to the annulus, because those three anchor launchers 40 are out of contact with corresponding tissue. The remaining seven anchor launchers 40 ARE positioned such that the respective anchor launcher can successfully drive the respective anchor into the annulus or tissue adjacent to the annulus, because those seven anchor launchers 40 are in contact (or at least close enough) with corresponding tissue.

Subsequent to the position adjusting, a first subset of the anchors 50 (i.e., those anchors which ARE positioned such that the respective anchor launcher can successfully drive the respective anchor into the annulus or tissue adjacent to the annulus) is identified (e.g., as described above in connection with FIGS. 3-5).

Subsequent to the identifying, a first subset of the anchor launchers (i.e., 7 anchor launchers 40 in the present example) that corresponds to the first subset of the anchors 50 is actuated. This causes the first subset of the anchor launchers 40 to drive the first subset of the anchors 50 into the annulus or tissue adjacent to the annulus, resulting in the state depicted in FIG. 16. In the present example, the total number of anchor launchers 40 is 10, and the first subset includes 7 anchors 50. This means that only 3 anchors 50 have not been launched at this point in the procedure.

Subsequent to the actuating of the first subset of the N anchor launchers, for each of the N anchors that have not yet been driven into the annulus or tissue adjacent to the annulus, catheter steering techniques are used to adjust a position of a respective corresponding anchor launcher to a position where the respective corresponding anchor launcher can successfully drive the anchor into the annulus or tissue adjacent to the annulus; and the respective corresponding anchor launcher is actuated so that the respective corresponding anchor launcher drives the anchor into the annulus or tissue adjacent to the annulus. The steps described in the following paragraphs is an example of this approach.

Subsequent to the actuating of the first subset of the N anchor launchers, the scaffold of arms that support the anchor launchers may collapse (fully or partially) because most of the anchors 50 that are attached to the flexible cord 30 are no longer held in place by their respective anchor launchers 40. Alternatively, in some embodiments, the scaffold of arms may be made from a soft metal that retains its shape after the majority of the anchors 50 have been launched. But the position of the anchor launchers 40 and the scaffold of arms that support the anchor launchers can still be adjusted using conventional catheter steering techniques. These conventional catheter steering techniques are used by the practitioner to manipulate the remaining anchor launchers 40 to corresponding desired positions (i.e., to positions where the corresponding anchors 50 can be driven into the annulus or adjacent tissue).

More specifically, subsequent to the actuating of the first subset of anchor launchers, (a) catheter steering techniques are used to move the entire assembly to a new position at which one or more of the unlaunched anchors is in a position where the respective anchor launcher 40 can successfully drive the unlaunched anchor(s) 50 into the annulus or tissue adjacent to the annulus, and (b) the respective corresponding anchor launcher(s) 40 is/are actuated so that the respective anchor launcher(s) 40 drives one or more of the unlaunched anchors 50 into the annulus or tissue adjacent to the annulus. For example, if three anchors 50 have not been previously launched, steps (a) and (b) in this paragraph might (i) bring only two of those three anchors into the suitable position and (ii) drive those two anchors into the annulus or tissue adjacent to the annulus. In this situation, steps (a) and (b) are repeated one additional time to bring the last anchor into the suitable position and drive the last anchor into the annulus or tissue adjacent to the annulus. If, on the other hand, the first occurrence of steps (a) and (b) in this paragraph successfully brought all three unlaunched anchors into the suitable position and drove all three of those anchors into the annulus or tissue adjacent to the annulus, there would be no need to repeat steps (a) and (b). Alternatively, if the first occurrence of steps (a) and (b) in this paragraph successfully brought only one of the unlaunched anchors into the suitable position and drove that one anchor into the annulus or tissue adjacent to the annulus, it might be necessary to repeat steps (a) and (b) two additional times.

The operator's control of the position of any given anchor in step (a) may rely on visual feedback obtained, for example, using echo imaging or fluoro imaging. Alternatively or additionally, the operator may rely on feedback based on signals received from a contact probe as described above in connection with the FIG. 3-5 embodiment.

Steps (a) and (b) are performed as many times as necessary until all of the anchors have been brought into a suitable position and driven into the annulus or tissue adjacent to the annulus. Finally, the anchor launchers 40 are withdrawn, leaving only the flexible cord 30, the optional sleeve 35 surrounding the flexible loop, and the anchors 50 (which have been driven into the annulus or adjacent tissue) behind in the patient's body, similar to the situation described above in connection with FIG. 9.

Notably, the extra slack in the flexible cord 30 and the optional sleeve 35 depicted in FIGS. 15 and 16 is helpful to prevent dislodgment of any anchors that have already been driven into the annulus or tissue adjacent to the annulus when catheter steering techniques are used to adjust the position of any anchor launchers that still contain unlaunched anchors. Optionally, similar slack may be added to the FIG. 3-5 embodiment described above.

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed:

1. A method for use at a tissue of a heart of a subject, the method comprising:
using a catheter, delivering to the heart a device that includes a cord and multiple anchors distributed along the cord;
subsequently, arranging the device in an arrangement at the tissue such that the cord is positioned and laid out correspondingly to the tissue;
while the device remains in the arrangement:
identifying a first subset of the anchors which are positioned for successful anchoring, and
subsequent to the identifying, for each of the anchors of the first subset, actuating a respective anchor launcher to drive the anchor into the tissue, those of the anchors that remain not driven into the tissue being undriven anchors; and subsequently, for each of the undriven anchors:
moving the undriven anchor to become positioned for successful anchoring, and
actuating a respective anchor launcher to drive the undriven anchor into the tissue.

2. The method according to claim 1, wherein:
the tissue is tissue of an annulus of a valve of the heart, and
arranging the device in the arrangement at the tissue comprises arranging the device such that the cord is positioned and laid out correspondingly to the annulus of the valve.

3. The method according to claim 1, wherein:
the device includes at least 10 anchors distributed along the cord, and
delivering the device to the heart comprises delivering to the heart the device that includes the at least 10 anchors distributed along the cord.

4. The method according to claim 1, wherein, for each of the anchor launchers, actuating the anchor launcher comprises pressing a respective launch button.

5. The method according to claim 1, wherein, for each of the anchor launchers, actuating the anchor launcher comprises actuating the anchor launcher by operating a solenoid.

6. The method according to claim 1, wherein, for each of the anchor launchers, actuating the anchor launcher comprises actuating the anchor launcher by operating a mechanical linkage.

7. The method according to claim 1, further comprising, once all of the anchors have been driven into the tissue, withdrawing the anchor launchers from the subject while the anchors and the cord remain at the tissue.

8. The method according to claim 1, further comprising, once all of the anchors have been driven into the tissue, withdrawing the catheter from the subject while the anchors and the cord remain at the tissue.

9. The method according to claim 1, further comprising, once all of the anchors have been driven into the tissue, contracting the tissue by tensioning the cord.

10. The method according to claim 1, wherein, for each of the undriven anchors, moving the undriven anchor comprises moving the undriven anchor using a manipulating tool.

11. The method according to claim 10, further comprising, via the catheter, advancing the manipulating tool to the device in the heart.

12. The method according to claim 10, wherein, for each of the undriven anchors, moving the undriven anchor comprises grabbing the undriven anchor using a grabber at a distal end of the manipulating tool.

13. The method according to claim 1, wherein, for each of the undriven anchors, moving the undriven anchor comprises moving the undriven anchor by steering the catheter.

14. The method according to claim 1, wherein the steps of (i) moving the undriven anchor, and (ii) actuating the respective anchor launcher to drive the undriven anchor into the tissue, are performed for each of the undriven anchors, successively.

15. The method of claim 1, wherein:
each of the anchors is slidably affixed with respect to the cord, and
arranging the device in the arrangement comprises sliding at least one of the anchors along the cord.

16. The method of claim 1, wherein identifying the first subset comprises identifying the first subset using an imaging technique.

17. The method of claim 1, wherein identifying the first subset comprises determining whether, for each of the anchor launchers, a respective contact probe that is affixed to the anchor launcher is in contact with the tissue.

18. The method of claim 1, wherein actuating the anchor launchers associated with the first subset of anchors comprises actuating each of the anchor launchers associated with the first subset of anchors substantially simultaneously.

19. The method of claim 1, wherein the arranging the device in the arrangement comprises inflating a balloon.

20. The method of claim 1, wherein the first subset includes at least two anchors.
